(19) United States
(12) Patent Application Publication
(10) Pub. No.: US 2025/0128075 A1
(43) Pub. Date: Apr. 24, 2025

(54) LEAD FAILURE DETECTION IN CARDIAC IMPLANTABLE DEVICES

(71) Applicant: Impulse Dynamics NV, Willemstad (CW)

(72) Inventors: Jason MEYERS, Haddonfield, NJ (US); David PRUTCHI, Voorhees, NJ (US)

(73) Assignee: Impulse Dynamics NV, Willemstad (CW)

(21) Appl. No.: 18/835,356

(22) PCT Filed: Feb. 2, 2023
(86) PCT No.: PCT/IB2023/050911

§ 371 Date: Aug. 2, 2024

Related U.S. Application Data
(60) Provisional application No. 63/305,707, filed on Feb. 2, 2022.

(51) Int. Cl. A61N 1/37; A61N 1/362; A61N 1/368; A61N 1/39

(52) U.S. Cl. CPC A61N 1/37; A61N 1/3627; A61N 1/36842; A61N 1/3925; A61N 1/39622

(57) ABSTRACT

A method for detecting lead failure in an implantable device using a multiple signal input configuration, including receiving a first signal from a first lead, receiving a second signal from a second lead, comparing the first signal and the second signal, and if the first signal is different from the second signal, determining whether one of the first lead and the second lead is faulty. Related apparatus and methods are also described.

### RELATED APPLICATION/S

This application claims the benefit of priority of U.S. Provisional Patent Application No. 63/305,707 filed Feb. 2, 2022, the contents of which are incorporated herein by reference in their entirety.

### FIELD AND BACKGROUND OF THE INVENTION

The present disclosure, in some embodiments thereof, relates to early lead failure detection in cardiac implantable electronic device systems, and, more particularly, but not exclusively, to early lead failure detection with multiple leads placed to sense a same signal, or multiple leads placed in a same chamber, or by multiple electrodes in the same lead, by tracking and comparing measurements from both leads or electrodes.

Cardiac implantable electronic device systems are often composed of an implantable cardiac device (for example an implantable pulse generator (IPG)) containing active electronics and connections for one or more transvenous leads. Examples of such systems may include, by way of some non-limiting examples, implantable cardiac devices which provide Cardiac Contractility Modulation therapy; pacemakers, which provide therapy for bradycardia; and Implantable Cardioverter/Defibrillators (ICD) which provide tachyarrhythmia therapy.

Lead failure is the most common failure mode in cardiac electronic implantable systems. In these systems, the leads are typically the point of failure, and it is critical to detect and correct these failures as quickly as possible to restore the availability of the therapy.

The disclosures of all references mentioned above and throughout the present specification, as well as the disclosures of all references mentioned in those references, are hereby incorporated herein by reference.

### SUMMARY OF THE INVENTION

The present disclosure, in some embodiments thereof, relates to early lead failure detection in cardiac implantable electronic device systems, and, more particularly, but not exclusively, to early lead failure detection with multiple leads placed to sense a same signal, or multiple leads placed in a same chamber, or by multiple electrodes in the same lead, by tracking and comparing measurements from both leads or electrodes.

According to an aspect of some embodiments of the present disclosure there is provided a method for detecting lead failure in an implantable device using a multiple signal input configuration, including receiving a first signal from a first lead, receiving a second signal from a second lead, comparing the first signal and the second signal, and if the first signal is different from the second signal, determining whether one of the first lead and the second lead is faulty.

According to some embodiments of the disclosure, the comparing includes processing the first signal is processed to produce a first processed signal, processing the second signal is processed to produce a second processed signal, and the first processed signal and the second processed signal are the signals which are compared.

According to some embodiments of the disclosure, the comparing includes processing the first signal is processed to produce a first R-wave signal, processing the second signal is processed to produce a second R-wave signal, and the first R-wave signal and the second R-wave signal are the signals which are compared.

According to some embodiments of the disclosure, the comparing includes performing a correlation between compared signals.

According to some embodiments of the disclosure, the comparing includes tracking changes in a correlation between compared signals.

According to some embodiments of the disclosure, the first lead and the second lead are both placed in a same cardiac chamber.

According to some embodiments of the disclosure, the first lead and the second lead are both placed in a right cardiac ventricle.

According to some embodiments of the disclosure, at least one of the first lead and the second lead are used to provide Cardiac Contractility Modulation treatment.

According to some embodiments of the disclosure, if the determining decides that one of the first lead and the second lead is faulty, producing an alert of potential lead failure.

According to some embodiments of the disclosure, including producing an alert of potential lead failure which includes which lead is suspect of having failed.

According to some embodiments of the disclosure, including sending the alert.

According to some embodiments of the disclosure, if a lead has been determined to be faulty, refraining from providing therapy.

According to some embodiments of the disclosure, if the first lead has been determined to be faulty, ignoring the signal from the first lead and using the second signal to determine whether to provide therapy.

According to some embodiments of the disclosure, if a lead has been determined to be faulty, refraining from providing therapy only via the lead which has been determined to be faulty.

According to some embodiments of the disclosure, if a lead has been determined to be faulty, refraining from providing therapy only via the lead which has been determined to be faulty and not refraining from providing therapy via a lead which has not been determined to be faulty.

According to some embodiments of the disclosure, the first lead and the second lead are connected to an implantable cardiac device, and if one of the first lead and the second lead has been determined to be faulty, refraining from providing therapy only via the lead which has been determined to be faulty.

According to some embodiments of the disclosure, the first signal and the second signal are intra-cardiac electrogram (IEGM) signals.

According to some embodiments of the disclosure, the comparing includes comparing over a specific time period.

According to some embodiments of the disclosure, the specific time period is selected from a group consisting of a specific number of seconds, a specific number of cardiac cycles, an interval between events in a cardiac cycle, a PQ interval, a QRS interval, a ST interval, a PR interval, and a RR interval.

According to some embodiments of the disclosure, the comparing the first signal and the second signal includes detecting R-waves in the first signal, detecting R-waves in the second signal, counting a first number of detected R-waves in the first signal during a specific time interval, counting a second number of detected R-waves in the second signal during the specific time interval, and comparing the first number and the second number.

According to some embodiments of the disclosure, if the first number is different from the second number by more than a specific value, determining that a lead associated with the greater number is faulty.

According to some embodiments of the disclosure, the comparing the first signal and the second signal includes detecting R-waves in the first signal, detecting R-waves in the second signal, measuring a difference in time between R-waves in the first signal and corresponding R-waves in the second signal, and if an average difference in time, as measured over a specific time interval, is greater than a specific threshold, then determining that one of the leads is faulty.

According to some embodiments of the disclosure, the comparing the first signal and the second signal includes tracking changes in a first IEGM signal and a second IEGM signal over time.

According to some embodiments of the disclosure, the tracking changes includes tracking IEGM signal noise during same cardiac intervals of a same lead at different times.

According to some embodiments of the disclosure, the comparing the first signal and the second signal includes comparing a level of signal noise in the first signal and a level of signal noise in the second signal.

According to some embodiments of the disclosure, if the level of signal noise in the first signal is greater than the level of signal noise in the second signal, determining that the first lead is faulty.

According to some embodiments of the disclosure, the comparing the first signal and the second signal includes producing a difference signal which includes a value of a difference between the first signal and the second signal, and determining whether one of the first lead and the second lead is faulty based on the difference signal.

According to some embodiments of the disclosure, the comparing the first signal and the second signal includes comparing impedance of the first lead and impedance of the second lead.

According to some embodiments of the disclosure, a lead with a greater impedance is determined to be faulty.

According to some embodiments of the disclosure, the receiving a first signal from the first lead comes from a first electrode on the first lead, and the receiving a second signal from the second lead comes from a second electrode on the first lead.

According to some embodiments of the disclosure, the comparing the first signal and the second signal includes tracking changes in the first signal and the second signal over time.

According to some embodiments of the disclosure, the comparing the first signal and the second signal includes tracking changes in impedance of the first lead and impedance of the second lead over time.

According to some embodiments of the disclosure, including making a first determination whether there is a cardiac problem based on the first signal, making a second determination whether there is a cardiac problem based on the second signal, and wherein the comparing includes comparing the first determination and the second determination.

According to an aspect of some embodiments of the present disclosure there is provided an implantable device including a first input for receiving a first signal, a second input for receiving a second signal, a module for comparing the first signal and the second signal, and a module for determining whether one of the first signal and the second signal is faulty.

According to some embodiments of the disclosure, the first input is for the first signal from a first electrode and the second input is for the second signal from a second electrode and the first electrode and the second electrode are both on a same lead.

According to some embodiments of the disclosure, the first input is for the first signal from a first lead and the second input is for the second signal from a second lead.

According to some embodiments of the disclosure, the module for comparing the first signal and the second signal is configured to compare intra-cardiac electrogram (IEGM) signals.

According to some embodiments of the disclosure, including a module for producing a difference signal including a difference between the first signal and the second signal, and the module for comparing the first signal and the second signal is configured to assess the difference signal.

According to some embodiments of the disclosure, including a module for measuring a first impedance of a lead connected to a first contact and a second impedance of a lead connected to a second contact.

According to some embodiments of the disclosure, the module for comparing compares the first impedance to the second impedance.

According to some embodiments of the disclosure, configured to produce R-wave signals and to compare timing of the R-wave signals.

According to some embodiments of the disclosure, configured to produce an alert if one of the first signal and the second signal is determined to be faulty.

According to some embodiments of the disclosure, configured to produce an alert including which of the first signal and the second signal is determined to be faulty.

According to some embodiments of the disclosure, the device includes an Implantable Cardioverter/Defibrillator (ICD).

According to some embodiments of the disclosure, the device includes a Cardiac Contractility Modulation device.

According to some embodiments of the disclosure, including a lead for providing Cardiac Contractility Modulation treatment.

According to some embodiments of the disclosure, the device includes a device selected from a group consisting of a Cardiac Contractility Modulation with ICD (CCM-D) combination device, a Cardiac Contractility Modulation with pacemaker combination device, and a Cardiac Contractility Modulation with pacemaker and ICD (CCM-D) combination device.

According to some embodiments of the disclosure, the device is configured to refrain from providing treatment if one of the first signal and the second signal is faulty.

According to some embodiments of the disclosure, the device is configured to refrain from providing treatment only via a faulty lead, and continue providing treatment via a non-faulty lead.

According to an aspect of some embodiments of the present disclosure there is provided a method for detecting lead failure in an implantable device using a multiple signal input configuration, including receiving a first signal from a first lead, receiving a second signal from a second lead, comparing the first signal and the second signal, and if the first signal is similar to the second signal, determining that neither one of the first lead and the second lead is faulty.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present disclosure may be embodied as a system, method or computer program product. Accordingly, some embodiments of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present disclosure may take the form of a computer program product embodied in one or more computer readable medium(s) having computer readable program code embodied thereon. Implementation of the method and/or system of some embodiments of the disclosure can involve performing and/or completing selected tasks manually, automatically, or a combination thereof. Moreover, according to actual instrumentation and equipment of some embodiments of the method and/or system of the disclosure, several selected tasks could be implemented by hardware, by software or by firmware and/or by a combination thereof, e.g., using an operating system.

For example, hardware for performing selected tasks according to some embodiments of the disclosure could be implemented as a chip or a circuit. As software, selected tasks according to some embodiments of the disclosure could be implemented as a plurality of software instructions being executed by a computer using any suitable operating system. In an exemplary embodiment of the disclosure, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as lead failure detection in cardiac implantable electronic device systems, might be expected to use completely different methods, e.g., making use of expert knowledge and/or the pattern recognition capabilities of the human brain, which may be different or even vastly more efficient than manually going through the steps of the methods described herein.

### BRIEF DESCRIPTION OF THE SEVERAL VIEWS OF THE DRAWING(S)

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIG. 1A is a simplified illustration of a single-lead cardiac implantable electronic device;

FIG. 1B is a simplified illustration of a cardiac implantable electronic device attached to two leads according to an example embodiment of the present disclosure;

FIG. 2A is a simplified block diagram illustration of modules in a multi-lead implantable cardiac device according to an example embodiment of the present disclosure;

FIG. 2B is a simplified block diagram illustration of modules in a multi-lead implantable cardiac device according to an example embodiment of the present disclosure;

FIG. 2C is a simplified block diagram illustration of modules in a multi-lead implantable cardiac device according to an example embodiment of the present disclosure; and

FIG. 3 is a simplified flow hart illustration of a method for detecting lead failure in implantable devices using a multiple lead configuration, according to an example embodiment of the present disclosure.

### DESCRIPTION OF SPECIFIC EMBODIMENTS OF THE INVENTION

The present disclosure, in some embodiments thereof, relates to early lead failure detection in cardiac implantable electronic device systems, and, more particularly, but not exclusively, to early lead failure detection with multiple leads placed to sense a same signal, or multiple leads placed in a same chamber, or by multiple electrodes in the same lead, by tracking and comparing measurements from both leads or electrodes.

Some non-limiting examples of implantable electronic device systems include an Implantable Cardioverter/Defibrillator (ICD) device and a Cardiac Contractility Modulation with ICD (CCM-D) device.

Some non-limiting examples of lead failure include lead fracture; conductor fracture; outer insulation failure; internal insulation failure and/or other types of lead failure. Lead failures may cause problems such as disconnects; shorts; intermittent shorts and/or a noisy signal.

The implantable electronic device systems may optionally monitor status of parameters associated with and/or signals received from input leads or electrodes on an input lead, and/or monitor parameters associated with output leads or electrodes on an output lead, and optionally determine whether the lead or electrode are apparently functioning well, or potentially faulty.

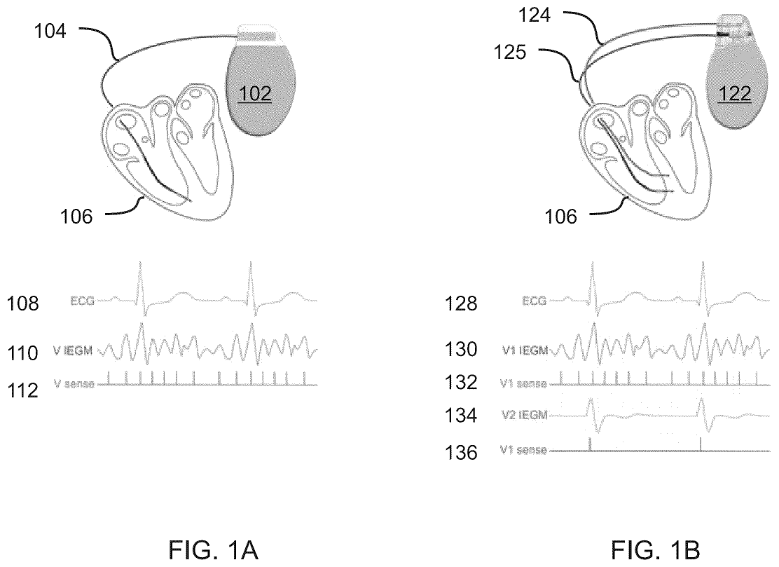

By way of a non-limiting example, for purposes of better understanding some embodiments of the present disclosure, reference is first made to the construction and operation of a cardiac implantable electronic device as illustrated in FIG. 1A.

Reference is now made to FIG. 1A, which is a simplified illustration of a single-lead cardiac implantable electronic device.

FIG. 1A shows a cardiac implantable electronic device 102 with one lead 104 implanted within a heart 106. FIG. 1A also shows an electrocardiogram (ECG) signal 108, aligned with an intra-cardiac electrogram (IEGM) signal 110 showing an electric signal sensed by a defective lead 104, and a signal 112 showing how the cardiac implantable electronic device 102 interprets the intra-cardiac electrogram signal 110. In FIG. 1A, the signal 112 shows timing of a detected R-wave. In an electrogram, a peak of the signal typically represents the R-wave. The signal 112 shows peaks in the intra-cardiac electrogram signal 110

In a case where the lead 104 is defective, a normal ECG signal 108 may be sensed as a noisy IEGM signal 110, and may be misinterpreted as ventricular activity (e.g. ventricular tachycardia or ventricular fibrillation). In some cases the misinterpretation may cause the cardiac implantable electronic device 102 to trigger inappropriate therapy such as shock.

The above-described potential problem has led to several major recalls of cardiac implantable electronic device leads.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

### INTRODUCTION

Cardiac implantable electronic devices may monitor parameters such as sensed signal amplitude(s), and/or pacing signals and/or Cardiac Contractility Modulation signals and/or impedance on each lead. If the monitored parameters change by more than a programmed amount, for example by more than a specific programmed percentage, an alert is optionally generated, to alert a physician to check the cardiac implantable electronic device system.

Such an approach has shortcomings, because, for example, sensed values such as R-wave amplitude and impedance may naturally vary significantly even when there is no failure, for example by a factor of 2 or more during initial weeks after implant, and by a factor of one third to one half over time after that. The variation may potentially be related to the time the device has been in a subject body after implantation. Because of the natural variation, selecting a good value for a percentage of change to trigger an alert, to both detect failures and reduce false alerts, is difficult. In some cases the percentage value is evaluated on a case-by-case basis.

In some embodiments, one or more of noise level, signal amplitude, signal shape and impedance are optionally evaluated based on comparison with historical values. In some embodiments, the comparison may be relative to a baseline generated during an initial time period, such as a first day, week month from device activation. In some embodiments, the comparison may be relative to reference values calculated every hour, day, week, month, and comparing a present value, or an average of some measured values, to a prior reference value.

Some cardiac implantable electronic device systems have two or more leads or electrodes placed close to each other, which potentially enables methods as described in the present disclosure.

Cardiac Contractility Modulation devices, for example, have two leads placed on the right ventricular septum, separated by approximately one or more centimeters, allowing a Cardiac Contractility Modulation with ICD (CCM-D) device to more easily identify lead conductor failures according to methods described herein, potentially preventing inappropriate shocks.

### Overview

An aspect of some embodiments relates to monitoring and/or comparing two or more signals from two or more leads or two or more electrodes on a lead, and detecting a potentially faulty lead or faulty electrode based on the comparison.

The term “lead” in all its grammatical forms is used throughout the present specification and claims interchangeably with the term “electrode” and its corresponding grammatical forms, so that descriptions related to two or more leads should be understood as potentially applying to two or more electrodes.

Where a comparison of two leads or signals or parameters related to two leads are described in the present specification, the description should be understood to include, mutatis mutandis, more than two leads.

In some embodiments, using two leads to measure physiological signals or physiological data causes an expectation that the signals or data from the two leads have some expected relationship between them.

In some embodiments, the relationship is expected to remain stable over time, or to change in a specific manner over time.

In some embodiments, when the relationship does not behave as expected, there is a likelihood that a reason for the unexpected may be a faulty lead.

In cases where two leads are expected to sense a same or a similar signal, the sensed signals are optionally compared. If the signals are different, that may be an indication that one of the two leads is potentially faulty.

Some non-limiting examples of cases where similar signals are expected include two leads placed close to each other in relation to an extent of a source of a signal, such as two leads in a same chamber in a heart, or two leads on a heart next to a same chamber.

In some embodiments, what is compared includes sensed electric signals, and/or various electric parameters related to the leads. Some non-limiting examples of what may be compared are described below, in a section named “Example comparisons”.

In some embodiments, a method a comparison includes tracking parameters such as, by way of some non-limiting examples, a relationship between two signals, such as a signal amplitude ratio, a ratio of noise in the signals, a time lag between the signals. If the parameter changes, or changes by a specific amount, one of the leads may be faulty.

In some embodiments, a difference between two leads at time of installation may indicate that one of the leads is faulty.

In some embodiments, a difference between two leads may optionally be recorded at a time of installation or at a specific time thereafter, and a subsequent change in the difference may indicate that one of the leads is faulty.

In some embodiments, a rapid change in the difference between two leads may be taken to indicate that one of the leads is faulty, and a gradual change may be disregarded, and/or recorded for subsequent use.

An aspect of some embodiments relates to a cardiac implantable electronic device systems reaching a determination based on input from one lead, and comparing the determination to a determination based on input from a second lead.

By way of a non-limiting example, a cardiac implantable electronic device system may determine that treatment for tachycardia may be desired, based on a noisy signal from a first lead. The system may optionally compare the determination to a similar determination from a second, not-noisy lead, and decide not to provide the tachycardia treatment.

By way of a non-limiting example, a cardiac implantable electronic device system may determine that Cardiac Contractility Modulation treatment should not be provided, because such treatment is provided in a non-refractory period, and based on a noisy lead from a first lead may not detect a non-refractory period. The system may optionally compare the determination to a similar determination from a second, not-noisy lead, detect a non-refractory period, and decide one or more of the following: produce an alert that the first lead is faulty; inhibit therapy until the device is checked by a clinician; and/or provide the Cardiac Contractility Modulation treatment.

An aspect of some embodiments relates to what is compared between signals of different leads or electrodes.

In some embodiments, what is compared may include one or more of the following:
signal frequency;
timing of a signal (e.g. timing of R-wave in an IEGM or an ECG signal, timing of other IEGM or ECG signal features);
signal amplitude;
a shape of an envelope of a signal;
a value of a parameter based on the shape; and
a power spectrum of a signal.

In some embodiments, what is compared includes detecting R-waves in a first signal from a first lead, detecting R-waves in a second signal from a second lead, and comparing the R-waves from the first lead and the second lead.

In some embodiments, if a difference in time of R-waves between the two leads is greater than 5, 10, 20, 50, 100, 150, 200 or 250 milliseconds, then one of the leads is optionally determined to be likely faulty.

In some embodiments, if an average difference in time, as measured over a specific time interval, between R-waves as sensed via the two leads is greater than 5, 10, or even up to 50 millisecond, then one of the leads is optionally determined to be likely faulty.

In some embodiments, what is compared is a number of R-wave detections in the first signal and the number of R-wave detections in the second signal, during a same time interval.

In some embodiments, if a difference between the number of detected R-wave is greater than 1, 2, 3 over a time period, a lead providing the greater number of R-wave detection is optionally determined to be likely faulty.

In some embodiments, if the number of detected R-waves from a first lead is greater than the number of detected R-waves from a second lead by a specific percentage, such as 1%, 5%, 10%, 20%, the first lead is optionally determined to be likely faulty.

In some embodiments, comparing signals may be performed by measuring similarity between the two signals, such as, by way of some non-limiting examples:
a shape of an envelope of the signals;
a ratio between the signal amplitudes;
a time difference between peaks in the signals (e.g. between R-waves in two ECG signals, and/or between other ECG signal features); and
a ratio between power spectra of the signals.

In some embodiments, comparing signals may be performed by calculating and/or tracking a transfer function between the two signals.

An aspect of some embodiments relates to what a cardiac implantable electronic device system does when a lead is determined to be likely faulty.

In some embodiments, the cardiac implantable electronic device system may perform one or more of the following:
record an alert that the lead may be faulty;
transmit an alert that the lead may be faulty;
cease using the lead to provide data for determining treatment;
cease using the lead for treatment; and
cease providing treatment.

When a cardiac implantable electronic device is connected to two or more leads, in cases of lead failure, there is great likelihood that only one lead will fail, and one or more other leads will not fail.

When a cardiac implantable electronic device is connected to two or more leads which potentially measure similar signals, it is possible to compare the two signals.

Comparison of two signals and/or parameters measured at two or more different leads potentially enables to detect that one of the signals is different from the other in a way which indicates that one of the signals is produced by a faulty lead.

### Example Comparisons

In some embodiments, when a signal from one lead indicates a healthy subject, and a signal from another lead indicates a non-healthy subject, it is determined that the subject is likely healthy, and that the lead producing the non-healthy signal is likely to be faulty. In some embodiments, one or more actions, as described below in a section named “Actions”, is taken by an implantable cardiac device based on detecting the difference between the leads.

In some embodiments, when a signal from one lead includes more electronic noise than a signal from another lead, it is determined that the noisy signal comes from a faulty lead. In some embodiments, one or more actions, as described below in a section named “Actions”, is taken by an implantable cardiac device based on detecting a likely-faulty lead.

In some embodiments, by way of a non-limiting example, measuring electronic noise may be done by measuring an RMS value of the signal measured. In some embodiments, noise can be calculated using RMS of the signal after high pass filter of the original signal. In some embodiments, when the measured noise is greater than greater than 1, 5, 10, 20, 50 or even 100% of the electronic signal, the signal may be considered noisy, and may indicate a potentially faulty lead.

In some embodiments, a signal from one lead is compared to a signal from another lead by evaluating a difference between the signals. When the difference is greater than a specific difference threshold, an optional determination that one of the leads is defective may be reached.

In some embodiments, a difference of more than 1, 5, 10, 20, 50 or even 100% of the electronic signal may indicate a potentially faulty lead.

In some embodiments, a difference in RMS value of the noise levels greater than 1, 5, or even 10% may indicate a potentially faulty lead.

In some embodiments, the noise level may be measured in signals after high pass filter of an original signal.

In some embodiments, a signal from one lead is compared to a signal from another lead by evaluating a change in a difference between the signals. When the change in the difference is greater than a specific difference change threshold, an optional determination that one of the leads is defective may be reached.

In some embodiments, a signal from one lead is compared to a signal from another lead by evaluating a value of cross-correlation between the signals, and/or a change over time in the value of the cross-correlation between the signals. When the value of the cross-correlation is greater than a specific threshold, and/or the change in value is greater than a specific change threshold, an optional determination that one of the leads is defective may be reached.

In some embodiments, a change in the cross correlation value which is greater than 1, 5, 10, 20, 50 or even 100% of the cross correlation value may indicate a potentially faulty lead.

In some embodiments, a change in QRS signal shape relative to a reference QRS signal shape may indicate a potentially faulty lead. In some embodiments, the reference QRS shape is a reference parameter, measured and optionally saved at intervals or times as described herein with reference to other reference parameters.

In some embodiments, a level of noise between the leads is optionally compared. In some embodiments, noise is optionally measured by using a high pass filter on the signals, such as, by way of some non-limiting examples, a high pass filter passing frequencies greater than 20 Hz, 50 Hz, 100 Hz. When the level of noise is greater than a specific threshold an optional determination that one of the leads is defective may be reached.

In some embodiments, a value of impedance as measured between two leads, and/or a change in the value of impedance between the leads, and/or a variance in value of impedance between the leads, is optionally compared. When one or more of the impedance values is greater than a corresponding threshold, an optional determination that one of the leads is defective may be reached.

In some embodiments, a change in the impedance measured between 2 leads of greater than 1, 5, 10, 20 50 or even 100% of the impedance may indicate a potentially faulty lead.

In some embodiments, a difference in value of impedance of the leads, and/or a change in the difference in value of impedance of the leads, and/or a variance in difference of value of impedance of the leads, is optionally compared. When the value of impedance or value of difference in value of impedance of the leads or variance in difference of value of impedance is greater than a corresponding threshold, an optional determination that one of the leads is defective may be reached.

In some embodiments, impedance of a lead is measured by dividing output voltage over the lead by output current through the lead.

In some embodiments, a difference signal is generated, showing a difference in signal value between a first lead and a second lead. When the difference signal is noisy, it is determined that at least one of the leads is faulty. In some embodiments, after detecting a noisy difference signal, an implantable cardiac device optionally measures noise from a first one of the two leads, and/or optionally measures noise from a second one of the two leads, and optionally determines whether one of the leads is producing most of the noise, in which case the noisy lead is determined to likely be faulty. In some embodiments, one or more actions, as described below in a section named “Actions”, is taken by an implantable cardiac device based on detecting a likely-faulty lead.

In some embodiments, a signal such as the signal 112 of FIG. 1A is produced by the implantable cardiac device for each one of the leads. If the signals from one lead appears to indicate a healthy subject, and the signal from another lead appears to indicate a non-healthy subject, it is determined that the subject is likely healthy, and that the lead producing the non-healthy signal is likely to be faulty. In some embodiments, one or more actions, as described below in a section named “Actions”, is taken by an implantable cardiac device based on detecting a likely-faulty lead.

In some embodiments, a signal such as the signal 112 of FIG. 1A is produced by the implantable cardiac device for each one of the leads. If the signals from one lead appear to indicate cardiac contraction at a time significantly different from a time indicated by another lead, it is determined that the at least one of the leads is likely to be faulty. In some embodiments, one or more actions, as described below in a section named “Actions”, is taken by an implantable cardiac device based on detecting a likely-faulty lead.

In some embodiments, a timing of peaks in a detected signal is produced by the implantable cardiac device for each one of the leads. A faulty lead may generate more noise than a non-faulty lead, and produce a distorted cardiac signal. The distorted cardiac signal may cause faulty detection of noise as an R-wave, and an incorrect detection of the R-wave timing. A faulty lead may cause a shift in detection in a range of 0.1 millisecond or more. In some embodiments, an absolute difference of R-wave timing is measured between 2 or more leads. If an absolute difference is greater than 2 milliseconds, it is an indication that there is a chance for a faulty lead. If the timing from one lead appears at a time significantly different from a time indicated by another lead, for example by more than 2 milliseconds or more, it is determined that the at least one of the leads is likely to be faulty. In some embodiments, when comparing timing of R-wave detection, it is enough to have one mismatch of R-wave timing detection every 1, 5, 10, 60 seconds may cause a determination that at least one of the leads is likely to be faulty. In some embodiments, one or more actions, as described below in a section named “Actions”, is taken by an implantable cardiac device based on detecting a likely-faulty lead.

In some embodiments, a timing of an R-wave is determined by the implantable cardiac device for each one of the leads. If the timing of the R-wave from one lead appears to be significantly different from a timing indicated by another lead, it is determined that the at least one of the leads is likely to be faulty. In some embodiments, one or more actions, as described below in a section named “Actions”, is taken by an implantable cardiac device based on detecting a likely-faulty lead.

In some embodiments, a change in QRS signal shape is compared to a reference QRS signal shape. A change of 10, 20, 50 or even 100% in a cross correlation calculation may be considered indicate a faulty lead.

In some embodiments, similar or even the same signal and/or measurements are taken from two or more leads over time. The signals and/or measurements are optionally compared. If changes in the signals and/or measurements change in a similar manner, also described as the signals and/or measurements tracking relative to each other, then a change in the signals/measurements is optionally not considered indicative of a faulty lead.

### What is Compared

In some embodiments raw signals coming from two or more leads are compared for differences as described above.

In some embodiments signals coming from two or more leads are compared following some pre-processing of the signals, such as R-wave detection and/or low pass filtering and/or high pass filtering. High pass filtering may be passing frequencies greater than a set frequency, where such a set frequency can be any value between 0.1-150 Hz. Typical values of set frequency are in a range between 1, 3, 5, 10, 20 Hz.

In some embodiments, high pass filtering is optionally used to detect a level of noise. A significant change in the RMS of a signal above a high pass filter frequency cutoff, for example, above 100 Hz, may indicate a faulty lead.

When using a high pass filter with a lower cutoff frequency, a signal that is being analyzed is typically not just an R-wave, which has high frequency content, but also other intervals in a cardiac signal, by way of a non-limiting example a PQ interval.

In some embodiments, R-wave signals such as the signal 112, based on measured signal 110 of FIG. 1A (and R-wave signals 132 and 136, based on measured signals 130 and 134 of FIG. 1B) are produced by the implantable cardiac device for each one of the leads, and the R-wave signals are compared.

In some embodiments, a time of cardiac contraction as detected by one lead is compared to a time of cardiac contraction as measured by another lead.

### Actions

In some embodiments, based on detecting a likely faulty lead, one or more of the following actions may be taken by the implantable cardiac device:
refraining from providing treatment, to avoid providing treatment potentially based on a signal from a faulty lead.
refraining from providing treatment via a lead which is suspected of being faulty.
producing an alert that there is likely to be a faulty lead.
producing an alert that there is likely to be a faulty lead, which alert includes an indication which lead is likely faulty.
sending an alert that there is likely to be a faulty lead.
sending an alert that there is likely to be a faulty lead, which alert includes an indication which lead is likely faulty.
changing treatment parameters based on ignoring a signal from a lead known or suspected of being faulty.

In some embodiments, similar or even the same signal and/or measurements are taken from two or more leads over time. The signals and/or measurements are optionally compared. If changes in the signals and/or measurements change in a similar manner, also described as the signals and/or measurements tracking relative to each other, then a change in the signals/measurements is optionally not considered indicative of a faulty lead, and optionally no action is taken.

Reference is now made to FIG. 1B, which is a simplified illustration of a cardiac implantable electronic device attached to two leads according to an example embodiment of the present disclosure;

FIG. 1B is intended to show how signals from two leads can be used to detect a faulty lead.

FIG. 1B shows a cardiac implantable electronic device 122 with a first lead 125 and a second lead 124 implanted within a heart 106. FIG. 1B also shows an electrocardiogram (ECG) signal 128, aligned with a first intra-cardiac electrogram (IEGM) signal 130 showing an electric signal sensed by a first defective lead 125, a first R wave signal 132 calculated from measured signal 130, showing how the cardiac implantable electronic device 122 may optionally interpret the first IEGM signal 130, a IEGM signal 134 showing an electric signal sensed by a not-defective lead 124, and a second R wave signal 136 calculated from the second IEGM signal 134, showing how the cardiac implantable electronic device 122 may optionally interpret the second IEGM signal 134. In FIG. 1B, the calculated R wave signal 132 shows timing of an erroneously interpreted R-wave in the IEGM signal 130, and the calculated R wave signal 136 shows timing of an R-wave in the IEGM signal 134.

In a case where the first lead 125 is defective, an actually normal ECG signal 128 may be sensed as a noisy IEGM signal 130, and may be misinterpreted, for example, as abnormal or unhealthy ventricular activity (e.g. ventricular tachycardia or ventricular fibrillation).

As shown in FIG. 1B, an example CCM-D implantable cardiac device 122 embodying the present invention can optionally correlate R-wave events detected on 2 leads—the first lead 125 and the second lead 124. If one of the leads is sensing events, for example occurrence of R-waves, differently than the other lead, that may optionally suggest that a rapid sequence of events is a result of lead conductor failure, potentially allowing the device to withhold inappropriate therapy, such as a cardiac shock, and/or to generate an alert.

Even if the sensed R-wave rate caused by intermittent conductor noise does not reach a tachycardia level, a similar concept can potentially enable early detection of lead failure, and optionally generate an alert.

It is noted that the alert can even identify which lead is likely to be faulty.

In some embodiments, the implantable cardiac device optionally generates an alert if the implantable cardiac device detects noise on only one of the two or more leads. In some embodiments, noise on two leads may optionally imply actual noise, and if noise is detected only on one lead, which may optionally suggest a likely lead failure.

Such methods and mechanisms as described herein can optionally be implemented in any device type that uses multiple cardiac leads, preferable intra-cardiac leads focusing on leads placed at the same chamber (e.g. Cardiac Contractility Modulation devices, multi-point CRT devices, etc.), and potentially enables a more robust detection of lead failures than the current methods.

The term lead, as used herein, refers implantable leads, such as intra-cardiac leads, for example defibrillation leads, with or without coils and/or sensing/pacing electrodes and/or pacing/sensing leads.

The electrode can be monopolar, bipolar or multi pole electrodes.

In some embodiments, in addition to correlating cardiac activity sensed by two or more leads or electrode pairs, lead failures, including lead fractures and lead dislodgements, may be identified by the implantable cardiac device periodically taking measurements of lead impedances and R-wave amplitudes. In some embodiments, values of the measurements are optionally compared, and the implantable cardiac device optionally confirm that, as the values change over time, the values change similarly. In some embodiments, an alert is optionally generated if the values begin to change significantly differently from each other.

In some embodiments, values of the measurements and/or values calculated based on the measurements, for parameters described herein such as impedance and/or noise, are saved in the implantable device and/or in an external record associated with a specific patient and/or a specific implantable device. In some embodiments, the values are optionally kept for each time interval of, for example, day, hour or minute. In some embodiments, the values are kept back for as long as a year, a month, a week, a day.

In some embodiments, such tracking of the measurement values potentially enables thresholds used to determine when a lead may be faulty to be tighter, that is, to have smaller measured parameter values, or smaller between-lead values, than is practical in a single-lead approach of prior-art devices, leading to earlier detection of lead failure. The tighter threshold values are potentially enabled based on comparing like with like-parameters for same patient, same device, rather than a population of patients.

In some embodiments, an allowable threshold for change of measurement values related to the leads over time may optionally be widened and/or eliminated, based on having signal from two or more leads to compare, potentially eliminating false lead failure detections.

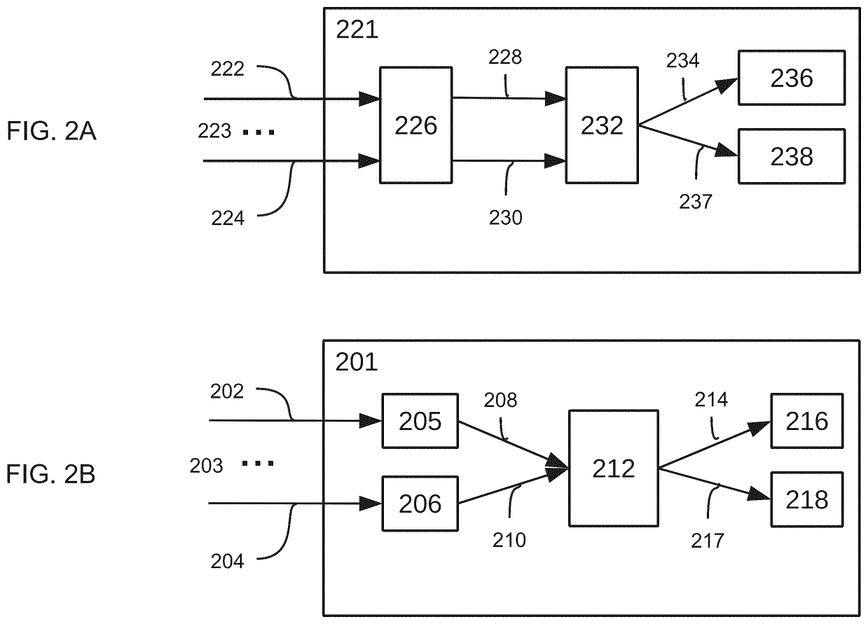

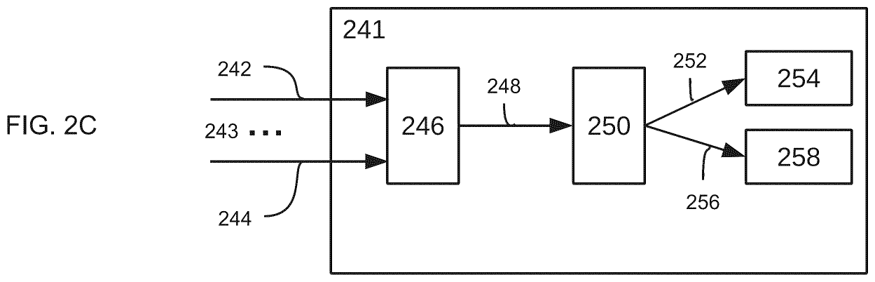

Reference is now made to FIG. 2A, which is a simplified block diagram illustration of modules in a multi-lead implantable cardiac device according to an example embodiment of the present disclosure.

FIG. 2A is intended to show an example of an implantable cardiac device which produces signals such as the R wave signals 132 136 shown in FIG. 1B, and potentially detects a faulty lead based on the signals.

FIG. 2A shows an implantable cardiac device 221 connected to two leads 222 224 or more leads 223. The leads provide input to one or more processor(s) 226, which produce signals 228 230 corresponding to, by way of a non-limiting example, the R wave signals 132 136 shown in FIG. 1B. The signals 228 230 are input to a signal comparing module 232, which compares the signals 228 230. The signal comparing module 232 is programmed to decide whether to send an alert 234 to a communication module 236, for optional communication that there is a faulty lead, and/or to send a decision 237 to additional components 238 within the implantable cardiac device 221 whether to initiate cardiac treatment by the implantable cardiac device 221, or to refrain from treatment, or which lead to use for treatment.

Reference is now made to FIG. 2B, which is a simplified block diagram illustration of modules in a multi-lead implantable cardiac device according to an example embodiment of the present disclosure.

FIG. 2B is intended to show an example of an implantable cardiac device which potentially detects a faulty lead based on the IEGM signals such as the IEGM signals 130 134 shown in FIG. 1B.

FIG. 2B shows an implantable cardiac device 201 connected to two leads 202 204 or more leads 203. The leads provide input to noise detectors 205 206, which either pass along 208 210 the IEGM signals received from the leads 202 204, or block one or both of the IEGM signals if they are detected to be noisy, therefore potentially unreliable.

A processing unit 212 receives the output of the noise detectors 205 206, and processes the received output, to decide whether to send an alert 214 to a communication module 216, for optional communication that there is a faulty lead, and/or to send a decision 217 to additional components 218 within the implantable cardiac device 201 whether to initiate cardiac treatment by the implantable cardiac device 201, or to refrain from treatment, or which lead to use for treatment.

Reference is now made to FIG. 2C, which is a simplified block diagram illustration of modules in a multi-lead implantable cardiac device according to an example embodiment of the present disclosure.

FIG. 2C is intended to show an example of an implantable cardiac device which potentially detects a faulty lead based on analyzing a difference between IEGM signals such as the IEGM signals 130 134 shown in FIG. 1B.

FIG. 2C shows an implantable cardiac device 241 connected to two leads 202 204 or more leads 203. The leads provide input to a module 246 for producing a difference signal 248.

A processing unit 250 receives the difference signal 248 and processes the received difference signal 248, to decide whether to send an alert 252 to a communication module 254, for optional communication that there is a faulty lead, and/or to send a decision 256 to additional components 258 within the implantable cardiac device 241 whether to initiate cardiac treatment by the implantable cardiac device 241, or to refrain from treatment, or which lead to use for treatment.

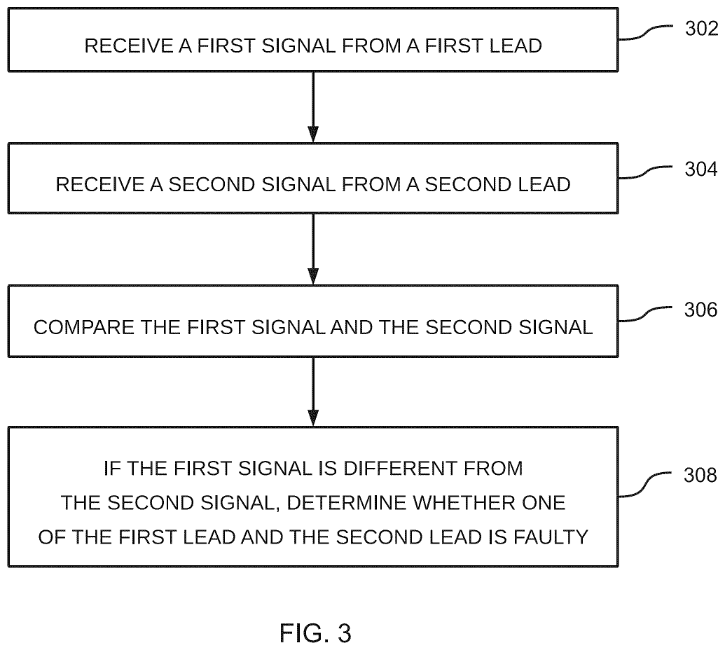

Reference is now mad to FIG. 3, which is a simplified flow hart illustration of a method for detecting lead failure in implantable devices using a multiple lead configuration, according to an example embodiment of the present disclosure.

The method shown in FIG. 3 includes:
receiving a first signal from a first lead (302);
receiving a second signal from a second lead (304);
comparing the first signal and the second signal (306); and
if the first signal is different from the second signal, determining whether one of the first lead and the second lead is faulty (308).

It is expected that during the life of a patent maturing from this application many relevant implantable cardiac devices will be developed and the scope of the term implantable cardiac device is intended to include all such new technologies a priori.

As used herein with reference to quantity or value, the term “about” means “within +10% of”.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method for detecting lead failure in an implantable device using a multiple signal input configuration, comprising:
receiving a first signal from a first lead;
receiving a second signal from a second lead;
wherein the first lead and the second lead are both placed in a same cardiac chamber,
comparing the first signal and the second signal; and
if the first signal is different from the second signal, determining whether one of the first lead and the second lead is faulty.

2. The method according to claim 1, wherein said comparing comprises:
processing the first signal is processed to produce a first processed signal;
processing the second signal is processed to produce a second processed signal; and
the first processed signal and the second processed signal are the signals which are compared.

3. The method according to claim 1, wherein said comparing comprises:
processing the first signal is processed to produce a first R-wave signal;
processing the second signal is processed to produce a second R-wave signal; and
the first R-wave signal and the second R-wave signal are the signals which are compared.

4. The method according to claim 1, wherein said comparing comprises performing a correlation between compared signals over a specific time period.

5-6. (canceled)

7. The method according to claim 1, wherein said cardiac chamber is a right cardiac ventricle.

8. The method according to claim 1, wherein at least one of the first lead and the second lead are used to provide Cardiac Contractility Modulation treatment.

9. The method according to claim 1, wherein if the determining decides that one of the first lead and the second lead is faulty, producing an alert of potential lead failure.

10-12. (canceled)

13. The method according to claim 1, wherein if the first lead has been determined to be faulty, ignoring the signal from the first lead and using the second signal to determine whether to provide therapy.

14-16. (canceled)

17. The method according to claim 1, wherein the first signal and the second signal are intra-cardiac electrogram (IEGM) signals.

18. (canceled)

19. The method according to claim 4, wherein the specific time period is selected from a group consisting of:
a specific number of seconds;
a specific number of cardiac cycles;
an interval between events in a cardiac cycle;
a PQ interval;
a QRS interval;
a ST interval;
a PR interval; and
a RR interval.

20. The method according to claim 1, wherein the comparing the first signal and the second signal comprises:
detecting R-waves in the first signal;
detecting R-waves in the second signal;
counting a first number of detected R-waves in the first signal during a specific time interval;
counting a second number of detected R-waves in the second signal during the specific time interval; and
comparing the first number and the second number.

21. The method according to claim 20, wherein if the first number is different from the second number by more than a specific value, determining that a lead associated with the greater number is faulty.

22. The method according to claim 1, wherein the comparing the first signal and the second signal comprises:
detecting R-waves in the first signal;
detecting R-waves in the second signal;
measuring a difference in time between R-waves in the first signal and corresponding R-waves in the second signal; and
if an average difference in time, as measured over a specific time interval, is greater than a specific threshold, then determining that one of the leads is faulty.

23. The method according to claim 1, wherein the comparing the first signal and the second signal comprises tracking changes in a first IEGM signal and a second IEGM signal over time.

24. (canceled)

25. The method according to claim 1, wherein the comparing the first signal and the second signal comprises comparing a level of signal noise in the first signal and a level of signal noise in the second signal.

26. (canceled)

27. The method according to claim 1, wherein the comparing the first signal and the second signal comprises producing a difference signal which comprises a value of a difference between the first signal and the second signal, and determining whether one of the first lead and the second lead is faulty based on the difference signal.

28. The method according to claim 1, wherein the comparing the first signal and the second signal comprises comparing impedance of the first lead and impedance of the second lead over time.

29-33. (canceled)

34. An implantable device comprising:
a first input for receiving a first signal from a first lead;
a second input for receiving a second signal from a second lead;
a comparison circuitry configured to align the first signal and the second signal; and
a determining circuitry configured to determine whether one of the first signal and the second signal is faulty based on a difference between said first signal and said second signal which are obtained from said first lead and said second lead when placed in the same cardiac chamber.

35. The device according to claim 34, wherein said first signal and said second signal are obtained over a specific time period.

36-46. (canceled)

47. The device according to claim 34, wherein the device comprises a device selected from a group consisting of:
a Cardiac Contractility Modulation with ICD (CCM-D) combination device;
a Cardiac Contractility Modulation with pacemaker combination device; and
a Cardiac Contractility Modulation with pacemaker and ICD (CCM-D) combination device.

48-49. (canceled)

50. A method for detecting lead failure in an implantable device using a multiple signal input configuration, comprising:
receiving a first signal from a first lead;
receiving a second signal from a second lead;
wherein said first lead and said second lead are both placed in a same cardiac chamber,
comparing the first signal and the second signal; and
if the first signal is similar to the second signal, determining that neither one of the first lead and the second lead is faulty.
